(19) United States
(12) Patent Application Publication
(10) Pub. No.: US 2015/0018722 A1
(43) Pub. Date: Jan. 15, 2015

(54) DETERMINATION, COMMUNICATION, AND PRESENTATION OF USER BODY POSITION INFORMATION

(71) Applicant: EZ AS A DRINK PRODUCTIONS, INC., Venice, CA (US)

(72) Inventor: Kostadin Dimitrov Yanev, Alamo, CA (US)

(21) Appl. No.: 13/937,959

(22) Filed: Jul. 9, 2013

(51) Int. Cl. A61B 5/00; A61B 5/0205; A61B 5/11

(52) U.S. Cl. CPC A61B 5/743; A61B 5/1118; A61B 5/0004; A61B 5/72; A61B 5/744; A61B 5/0205; A61B 5/1121
USPC 600/595

(57) ABSTRACT

This disclosure relates to a system configured for determination, communication, and presentation of body positions of users. The system may facilitate communication related to body positions of a first user between the first user and one or more remotely located second users and/or systems. The communication may include verbal communication, communication of visual images and/or videos, and/or other communication such that the second user may provide real-time, near real-time, and/or later feedback to the first user. The system may allow the one or more second users and/or systems, to locally and/or remotely provide coaching, a medical diagnosis, care related information, physical therapy rehabilitation, interaction, and/or other services to the first user. A given body position may describe a spatial position of one or more body parts of a user. The system may comprise body position sensors, client computing platforms, a server, external resources, and/or other components.

### FIELD OF THE DISCLOSURE

This disclosure relates to systems and methods for determination, communication, and/or presentation of information related to body positions of users.

### BACKGROUND

Various health and/or fitness monitoring systems exist. Examples of existing health and fitness monitoring systems include wearable devices that transmit physiological data (e.g., heart rate, respiration rate) related to a specific health condition, systems that facilitate exercise as part of an electronic game, and systems configured to collect and record data from multiple in-home monitoring devices (e.g., a scale) and/or tests (e.g., a blood glucose test) such that the collected data may be monitored over time.

### SUMMARY

One aspect of the disclosure relates to a system configured for determination, communication, and presentation of information related to body positions of users, in accordance with one or more implementations. Information related to body positions of a first user may be communicated between the first user and one or more remotely located users and/or systems. The communication may include verbal communication, communication of visual images and/or videos, and/or other communication such that a second user may, in real-time, near real-time, and/or at a later time, observe body positions of the first user and/or provide feedback to the first user. The system may allow one or more remotely-located users and/or systems to provide coaching, a medical diagnosis, care related information, physical therapy rehabilitation, and/or other wellness-related services and/or interactions to the first user. In some implementations, interactions provided to the first user may include game play and/or other interactions. In some implementations, the remotely-located users may include a coach, a doctor, a care giver, a physical therapist, a fellow trainee, a fellow patient, a fellow player, an insurance provider, an employer, an agent of an employer, and/or other users. In some implementations, “remote” may describe physical distance between client computing platforms. The physical distance may be relatively small (e.g., next to each other, in the next room) and/or relatively large (e.g., in another city or country).

A given body position may describe a spatial position of one or more body parts of a user. The system may be configured to receive body position signals conveying body position information associated with one or more body positions of the first user. The body position signal may be received directly or indirectly from a body position sensor associated with the first user. The body position information may be received responsive to physical activity performed by the first user, and/or at other times. The body position sensor may be included in a first client computing platform associated with the first user. The system may be configured such that the body position information is parameterized by a processor of the first client computing platform and then transmitted to a server. The server may be configured to generate presentation information related to the body positions of the first user for presentation to the first user, a second user (who may be remotely located from the first user), and/or other users. The presentation information may be determined based on the parameterized information transmitted to the server. In some implementations, the system may comprise one or more body position sensors, one or more client computing platforms, the server, external resources, and/or other components.

The body position sensor may be configured to provide one or more body position signals conveying information associated with one or more body positions and/or physical characteristics of the first user. The body position signals may be provided responsive to physical activity performed by the first user.

The client computing platforms may include one or more processors, one or more body position sensors, a user interface, electronic storage, and/or other components. The processor may be configured to execute computer program modules. The computer program modules may be configured to enable an expert or user associated with a given client computing platform to interface with the system and/or the external resources, and/or provide other functionality attributed herein to the client computing platforms.

The processor of a client computing platform may be configured to execute one or more computer program modules. The computer program modules may include one or more of a body recognition module, a parameterization module, a client communication module, a client presentation module, and/or other modules.

The body recognition module may be configured to receive the body position signal conveying body position information associated with one or more body positions of the first user from the body position sensor. The body position signal may be received directly and/or indirectly from the body position sensor.

The parameterization module may be configured to parameterize the body position information associated with the first user to provide parameterized body position information. The parameterized body position information may be related to the body positions and/or physical characteristics of the first user. The parameterized body position information may comprise less electronic space than the corresponding body position information conveyed by the body position signal. For example, the parameterized body position information may comprise less bytes than the body position information conveyed by the body position signal. In some implementations, the parameterization module may be configured such that parameterizing includes determining one or more parameters related to the body positions and/or physical characteristics of the first user.

The client communication module may be configured to transmit and receive information. For example, the client communication module may be configured to transmit a parameterized information signal conveying the parameterized body position information. The client communication module may be configured to transmit the parameterized information signal to the server and/or other devices. Client communication module 26 may be configured to transmit the parameterized information signal conveying the parameterized body position information in real-time, near real-time, and/or at a later time.

The server may be configured to execute one or more computer program modules. The computer program modules may include one or more of a user module, a server communication module, an analysis module, a virtualization module, and/or other modules.

The user module may be configured to access and/or manage one or more user profiles and/or user information associated with users of the system. The user profiles may include, for example, information identifying users within the system, account information, information related to relationships between users (e.g., doctor/patient), interaction history among users of the system, a client computing platform identification associated with a user, and/or other information related to users.

The server communication module may be configured to transmit and/or receive information. For example, the server communication module may be configured to facilitate receiving the parameterized information signal conveying parameterized body position information associated with one or more body positions of the first user.

The analysis module may be configured to analyze the parameterized information signal to determine information related to the one or more body positions and/or physical characteristics of the first user, analyze the parameters determined by the parameterization module, and/or perform other functions. In some implementations, the analysis module may be configured to identify individual body parts, physical characteristics of individual body parts, an overall body position of the first user, overall physical characteristics of the first user, a physical activity performed by the first user, and/or other information associated with the first user.

The virtualization module may be configured to generate presentation information for presentation to the first user and/or other users. The presentation information may be generated based on the received parameterized information signal, the analysis by the analysis module, and/or other information. The presentation information may be presented to the first user via a client computing platform associated with the first user. The presentation information may be presented to other remotely-located users via client computing platforms associated with the other users. The presentation information may be associated with the body position, physical characteristics, and/or movement (e.g., physical activity) of the first user.

Referring again to the client communication module, in some implementations, the client communication module may be configured to facilitate receiving the presentation information (e.g., from the server) for presentation to a given user via a given client computing platform associated with the given user. The client presentation module may be configured to facilitate presentation of the received presentation information on the given client computing platform via the user interface.

In some implementations, the client presentation module may be configured such that users may manipulate views of the presented information. The users may manipulate the presented information via gestures and/or other information entered into the user interface.

In some implementations, the server communication module and/or the client communication module may be configured to facilitate bi-directional communication between client computing platforms associated with the first user and one or more second users. The server communication module and/or the client communication module may be configured such that the first user and/or the one or more second users may communicate information related to body parts, movements and/or other actions by the first user, parameters, and/or other information to each other.

These and other objects, features, and characteristics of the system and/or method disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured for determination, communication, and presentation of information related to body positions of users, in accordance with one or more implementations.

FIG. 2 illustrates a view of information associated with the body position, physical characteristics, and/or movement of a user, in accordance with one or more implementations.

FIG. 3 illustrates a method for real-time determination, communication, and presentation of information related to body positions, in accordance with one or more implementations.

### DETAILED DESCRIPTION

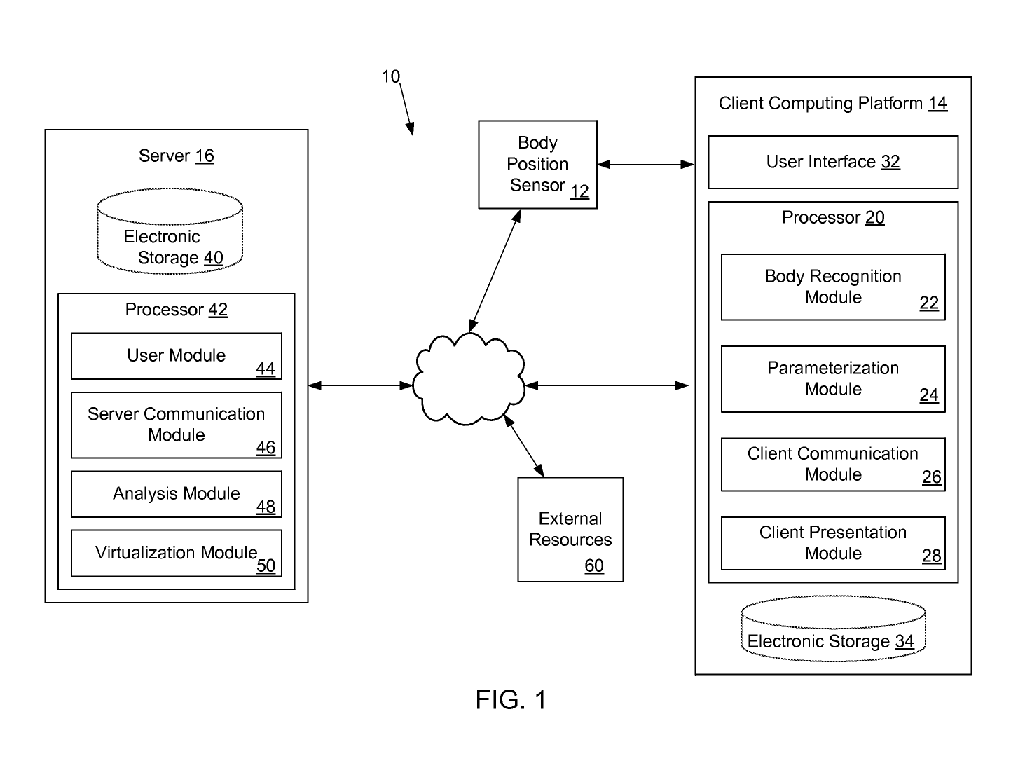

FIG. 1 illustrates a system 10 configured for determination, communication, and presentation of information related to body positions of users, in accordance with one or more implementations. In some implementations, system 10 may comprise one or more body position sensors 12, one or more client computing platforms 14, a server 16, external resources 60, and/or other components. In some implementations, server 16 may be configured to communicate with one or more client computing platforms 14 and/or body position sensors 12 according to a client/server architecture. In some implementations, a given client computing platform 14 may be configured to communicate with other client computing platforms 14 according to a peer-to-peer architecture. The users may access system 10 via client computing platforms 14.

System 10 may be configured to determine, communicate, analyze, and present the body positions of users in real-time, near real-time, and/or at a later time. For example, the information determined, communicated, and/or presented may be stored for later analysis and/or presentation. In some implementations, the stored information may be compared to current information, other previously determined information (e.g., historical data), and/or other information.

In some implementations, system 10 may be configured for use in conjunction with an exercise system such as, for example, those described in U.S. patent application Ser. No. 13/527,465 filed Jun. 19, 2012 and entitled “Personal Wellness Device”, U.S. patent application Ser. No. 13/403,803 filed Feb. 23, 2012 and entitled “Personal Exercise Device”, and/or U.S. patent application Ser. No. 12/818,977 filed Jun. 18, 2010 and entitled “Modular Exercise System”, all of which are incorporated herein by reference. Using system 10 in conjunction with one or more of the exercise systems described in the patent applications listed above may include, for example, parameterizing one or more body positions and/or physical characteristics of the first user during exercise while operating one of the exercise systems described above. Information determined by the exercise system may be correlated with one or more body positions of the first user.

Body position sensor 12 may be configured to provide one or more body position signals conveying information associated with one or more body positions and/or physical characteristics of a first user. The body position signals may be provided responsive to physical activity performed by the first user. A given body position may describe, for example, a spatial position, orientation, posture, and/or other positions of the first user and/or of one or more body parts of the first user. A given physical characteristic may include, for example, a size, a length, a weight, a shape, and/or other characteristics of the first user, and/or of one or more body parts of the first user. The body position signals conveying information associated with the body positions and/or physical characteristics of the user may include measurement information related to the physical size, shape, weight, and/or other physical characteristics of the first user. The one or more body parts of the first user may include a portion of the first user's body (e.g., one or more of a foot, hand, head, arm, leg, and/or other body parts).

Physical activity performed by the user may include any movement, motion, and/or other activity performed by the user. Physical activity may include exercise, normal daily activities, and/or other physical activities. Exercise may include, for example, hiking, running, biking, stretching, yoga, playing a team sport, weightlifting, calisthenics, isometrics, and/or other exercises. Normal daily activities may include household chores, commuting, working at a computer, shopping, making a meal, and/or other normal daily activities. In some implementations, physical activity may include maintaining a given posture for a period of time. For example, physical activity may include sitting, standing, lying down, and/or maintaining other postures for a period of time.

In some implementations, the information conveyed by the body position signals provided by body position sensor 12 may include visual information representing the first user. The visual information representing the first user may include one or more of still images, video images, and/or other information. For example, body position sensor 12 may be configured to provide body position signals conveying information associated with the position of one or more body parts of the first user relative to each other. According to some implementations, body position sensor 12 may include an infrared stereoscopic sensor configured to facilitate determination of user body positions, such as for example the Kinect™ available from Microsoft™ of Redmond, Wash.

In some implementations, information conveyed by the body position signals provided by body position sensor 12 may include information other than visual information associated with the one or more body positions and/or physical characteristics of the first user. For example, body position sensor 12 may be configured to provide body position signals conveying information related to one or more body positions and/or physical characteristics of the first user based on one or more contact locations of the first user on a weight sensitive surface. For example, the weight sensitive surface may include a weight sensitive floor mat configured to provide output signals conveying information related to the positions of the feet of the user on the floor mat, and/or the weight of the user. As another example, body position sensor 12 may be configured to provide body position signals conveying information related to the geographic location of the first user. Changes in the physical location of the first user may indicate physical activity, for example. In some implementations, body position sensor 12 may be built into exercise equipment. Examples of sensors suitable for inclusion in system 10 as body position sensor 12 include one or more of a 3D scanner, an RGB camera, an infrared projector and camera, a CMOS image sensor, mechanical transducers, piezoelectric transducers, capacitive transducers, proximity sensors, microphones, a multi-array microphone, GPS devices, accelerometer, magnetometer, and/or other sensors.

In some implementations, body position sensor 12 may be separate from a given client computing platform 14 and communicate with client computing platform 14 as a peripheral device. The body position sensor 12 may be integrated with a given client computing platform 14 as a single device. In some implementations, body position sensor 12 and/or the client computing platform 14 associated with the first user may be carried by the first user. For example, body position sensor 12 may be included in a Smartphone associated with the first user. As such, information related to body positions and/or physical activity of the first user may be obtained throughout the day as the first user goes about his daily business and/or participates in specific activities.

Although body position sensor 12 is depicted in FIG. 1 as a single element, this is not intended to be limiting as other implementation that include multiple body position sensors 12 are contemplated and within the scope of the disclosure. For example, in some implementations, a given client computing platform 14 may have one or more body position sensors 12 integrated therewith, and/or be in communication with one or more body position sensors 12 as separate peripheral devices.

Client computing platforms 14 may include one or more processors 20, one or more body position sensors 12 (as discussed above), a user interface 32, electronic storage 34, and/or other components. Processor 20 may be configured to execute computer program modules. The computer program modules may be configured to enable an expert or user associated with a given client computing platform 14 to interface with system 10 and/or external resources 60, and/or provide other functionality attributed herein to client computing platforms 14. By way of non-limiting example, a given client computing platform 14 may include one or more of a desktop computer, a laptop computer, a handheld computer, a tablet computing platform, a NetBook, a Smartphone, a gaming console, a personal wellness device similar to the one incorporated by reference above, and/or other computing platforms.

Processor 20 of a client computing platform 14 may be configured to execute one or more computer program modules. The computer program modules may include one or more of a body recognition module 22, a parameterization module 24, a client communication module 26, a client presentation module 28, and/or other modules.

Body recognition module 22 may be configured to receive the body position signal conveying body position information associated with one or more body positions of the first user from body position sensor 12. The body position signal may be received directly and/or indirectly from body position sensor 12. For example, body position sensor 12 may be built into exercise equipment and the body position signal may be transmitted directly from body position sensor 12 to body recognition module 22. As another example, body position 12 may be a camera in a gaming system. The body position signal may be transmitted from the camera to an intermediate computing device (e.g., the game console) before being received by body recognition module 22.

Parameterization module 24 may be configured to parameterize the body position information associated with the first user to provide parameterized body position information. The parameterized body position information may be related to the body positions and/or physical characteristics of the first user. The parameterized body position information may comprise less electronic space than the corresponding body position information conveyed by the body position signal. For example, the parameterized body position information may comprise less bytes than the body position information conveyed by the body position signal.

In some implementations, parameterization module 24 may be configured such that parameterizing includes determining one or more parameters related to the body positions and/or physical characteristics of the first user. A given parameter may convey numerical information associated with a corresponding body position and/or physical characteristic. In some implementations, the parameterized body position information may convey information used to identify the physical activity performed by the first user. In some implementations, parameterization may include a mathematical process involving the identification of a complete or reduced set of effective coordinates or degrees of freedom of the user and/or body parts of the user. For example, parameterization of a line, surface, or volume may imply identification of a set of coordinates that allows one to uniquely identify any point (on the line, surface, or volume) with an ordered list of numbers. Individual ones of the coordinates may be defined parametrically in the form of a parametric curve (one-dimensional) or a parametric equation (two or more dimensions).

In some implementations, the parameterized body position information may convey one or more of an absolute position, a relative position, a coordinate position, an angle, a geographic location, a velocity, a force, a timing, a duration, a distance, a respiration rate, an effort, a route, a pace, a heart rate, a number of steps, a trajectory, a volume parameter, an area parameter, an orientation parameter, a rhythm parameter, a size, a parameter describing the relative position of two or more body parts (e.g., head, torso, legs, arms, hands, feet, and/or other body parts), a width, a weight, a depth, and/or other parameters. For example, the parameterized body position information may convey an angle between the first user's shoulder and wrist, a waist size, a respiration rate, a heart rate, a depth related to the distance of the first user from a real or virtual reference plane, and/or other parameters.

Client communication module 26 may be configured to transmit and receive information. For example, client communication module 26 may be configured to transmit a parameterized information signal conveying the parameterized body position information. Client communication module 26 may be configured to transmit the parameterized information signal to server 16, and/or other devices. Client communication module 26 may be configured to transmit the parameterized information signal conveying the parameterized body position information in real-time, near real-time, and/or at a later time.

The parameterized body position information may be used by server 16, client computing platforms 14, and/or the other devices to generate analytical and/or presentation information for presentation to the first user and/or one or more other users via client computing platforms 14. The one or more other users may include the second user, for example, who may be remotely located from the first user. The presentation information may include a visual representation of the one or more body positions of the first user, textual information associated with the one or more body positions of the first user, graphical information associated with the one or more body positions of the first user, and/or other information. The presentation information is described in greater detail below.

Server 16 may be configured to execute one or more computer program modules. The computer program modules may include one or more of a user module 44, a server communication module 46, an analysis module 48, a virtualization module 50, and/or other modules.

User module 44 may be configured to access and/or manage one or more user profiles and/or user information associated with users of system 10. The one or more user profiles and/or user information may include information stored by server 16, one or more of the client computing platforms 14, and/or other storage locations. The user profiles may include, for example, information identifying users (e.g., a username or handle, a number, an identifier, and/or other identifying information) within the system, security login information (e.g., a login code or password), account information, subscription information, information related to relationships between users (e.g., doctor/patient), demographic information associated with users, medical information associated with users, interaction history among users of system 10, a client computing platform 14 identification associated with a user, a phone number associated with a user, analyzed and/or historical information related to the user's physical and/or physiological health, information related to the user's previous and/or current physical activity, two-dimensional and/or three-dimensional pictures and/or representations of the users, avatars associated with the users, and/or other information related to the users.

Server communication module 46 may be configured to transmit and/or receive information. For example, server communication module 46 may be configured to facilitate receiving the parameterized information signal conveying parameterized body position information associated with one or more body positions of the first user. Server communication module 46 may facilitate receiving the parameterized information signal via communication hardware, software, and/or other equipment. Communication hardware and/or software may include, for example, antennas, routers, modems, transceivers, encoders, hardware and software used by a cellular communication network, hardware and software used by a WiFi network, and/or other equipment.

Analysis module 48 may be configured to analyze the parameterized information signal to determine information related to the one or more body positions and/or physical characteristics of the first user, analyze the parameters determined by parameterization module 24, and/or perform other functions. In some implementations, analysis module 48 may be configured to identify individual body parts, physical characteristics of individual body parts, an overall body position of the first user (e.g., a posture), overall physical characteristics of the first user, and/or other information associated with the first user. Analysis module 48 may be configured to identify spatial positions and/or physical characteristics of one or more body parts relative to each other. In some implementations, analysis module 48 may be configured to identify separate body positions and/or physical characteristics for more than one user at a time.

Analysis module 48 may be configured to identify the one or more body positions and/or physical characteristics of the first user by interpreting the parameterized information signal via mathematical algorithms. The mathematical algorithms may include three-dimensional model-based algorithms, skeletal-based algorithms, appearance-based algorithms, and/or other algorithms configured to interpret the signals conveying user body positions and/or physical characteristics information.

In some implementations, the analysis by analysis module 48 may include performing calculations based on one or more calculation methods. In some implementations, the calculation methods may be determined at manufacture, inputted into system 10 by a user via client computing platform(s) 14, and/or determined by other methods. In some implementations, instructions to perform the calculation methods may be stored by server 16, by client computing platform(s) 16, external resources 60, and/or other devices. In some implementations, analysis module 48 may be configured to associate determined parameters with a date, a time of day, and/or other time related information. In some implementations, analysis module 48 may be configured to determine changes in one or more parameters. For example, analysis module 48 may be configured to analyze changes in waist size. As another example, analysis module 48 may be configured to determine an average angle based on data from one or more users. In some implementations, the results of analysis may be output to other modules (e.g., virtualization module 50) and/or systems (e.g., client computing platforms 14, external resources 60).

In some implementations, analysis module 48 may be configured to determine information related to the movement of the first user. Analysis module 48 may be configured to determine the information associated with movement based on the parameterized information signal, and/or based on other information. Information associated with the movement of the first user may include, for example, information related to a velocity, a timing, a duration, a distance, changes in a geographic location, and/or other parameters.

In some implementations, the determination of the information related to the movement of the first user may include a determination that the first user is moving. Movement of the first user may include a change in the one or more body positions of the first user within a given period of time. A change in the one or more body positions of the first user may include a change in the spatial position of the first user's body, a change in the spatial position of a portion of the first user's body (e.g., torso and head), and/or a change in the spatial position of one or more body parts of the first user. In some implementations, analysis module 48 may be configured to make a determination of movement based on successive body positions determined by analysis module 48. Analysis module 48 may be configured to compare a first body position at a first time point to a second body position at a second time point.

In some implementations, analysis module 48 may be configured to analyze the parameterized information signal and identify the physical activity performed by the first user. Analysis module 48 may identify the physical activity performed by the first user based on the parameterized body position information communicated in the parameterized information signal, the identified spatial positions of one or more body parts, the analysis via the mathematical algorithms, the determination of the information related to movement, and/or other information. For example, analysis module 48 may be configured to identify whether the first user is walking, doing pushups, sitting at a desk, and/or performing other physical activities.

In some implementations, analysis module 48 may be configured to determine whether the first user is performing a given physical activity in a prescribed manner. Analysis module 48 may be configured to compare the analyzed information related to the given physical activity to a target and/or a reference for the given physical activity. Based on a comparison between the analyzed information and the target and/or reference, analysis module 48 may be configured to provide feedback to the first user and/or to other users. Such feedback may include one or more of coaching, warnings, incentives, and/or other feedback. The target and/or reference for the given physical activity may be provided by another user via a client computing platform 14, obtained by analysis module 48 via external resources 60, and/or provided in other ways. For example, analysis module 48 may be configured to determine whether the first user's hands are in a correct position while the first user performs pushups.

Virtualization module 50 may be configured to generate presentation information for presentation to the first user, the second user, and/or other users. The presentation information may be generated based on the received parameterized information signal, the analysis by analysis module 48, the users' profile data and/or other information. The presentation information may be presented to the first user via a client computing platform 14 associated with the first user. The presentation information may be presented to other remotely-located users via client computing platforms 14 associated with the other users.

The presentation information may be associated with the body position, physical characteristics, and/or movement (e.g., physical activity) of the first user. The presentation information may convey one or more of the parameters parameterized by parameterization module 24, results of the analysis performed by analysis module 48, and/or other information. The presentation information may include a visual representation of the body position, physical characteristics, and/or movement of the first user; textual information associated with the body position, physical characteristics, and/or movement of the first user; graphical information associated with the body position, physical characteristics, and/or movement of the first user; and/or other information.

In some implementations, the visual representation of the body position, physical characteristics, and/or movement of the first user may include an avatar configured to represent the first user. Virtualization module 50 may be configured to present the avatar to the first user, the second user, and/or other users via client computing platforms 14. Virtualization module 50 may be configured to cause the avatar to express the body position, physical characteristics, and/or movements of the first user. Virtualization module 50 may be configured to cause the avatar to move in a view presented to the first user, the second user, and/or other users responsive to the first user moving and/or changing body position. In some implementations, the avatar may be presented in real-time and/or at another time. In some implementations, the textual information associated with the body position, physical characteristics, and/or movement of the first user may include a textual listing of the determined parameters, and/or other textual information. In some implementations, the graphical information associated with the body position, physical characteristics, and/or movement of the first user may include graphs, meters, dials, and/or other graphical information associated with the body position, physical characteristics, and/or movement of the first user.

In some implementations, virtualization module 50 may be configured such that the presentation information may include information related to a target body position of the first user, and/or information related to an actual body position of the first user. The actual body position may be the current body position and/or physical characteristics of the first user as described above. The target body position may comprise a target position of one or more body parts and/or physical characteristics. In some implementations, virtualization module 50 may be configured such that the first user, the second user, and/or the other users may be able to control the target body position via client computing platforms 14. Virtualization module 50 may be configured to receive control information from the users via client computing platforms 14 indicating target positions for one or more body parts of the first user. In some implementations, virtualization module 50 may be configured such that the presentation information related to the target body position is generated and/or displayed based on information determined by analysis module 48, communication facilitated by client communication module 26 and/or server communication module 46, and/or other information.

In practice, the second and/or other users may utilize system 10 to communicate a target body position and the first user may attempt to match his body position to the target body position. In some implementations, the presentation information related to the target body position and/or the actual body position may be displayed separately and/or at the same time. In some implementations, the presentation information related to the actual body position may be overlaid on the presentation information related to the target body position and/or vice versa.

By way of a non-limiting example, virtualization module 50 may be configured such that a physical therapist located remotely from the first user may view information associated with a range of motion of one or more body parts of the first user. The physical therapist may be able to view a visual representation (via an avatar) of the first user's neck, a numerical angular measurement between the first user's tilted head and the first user's shoulders, a chart showing the first user's neck flexibility over time, and/or other information. The physical therapist may be able to communicate a target neck position to the first user and analyze whether the first user conforms to the target neck position.

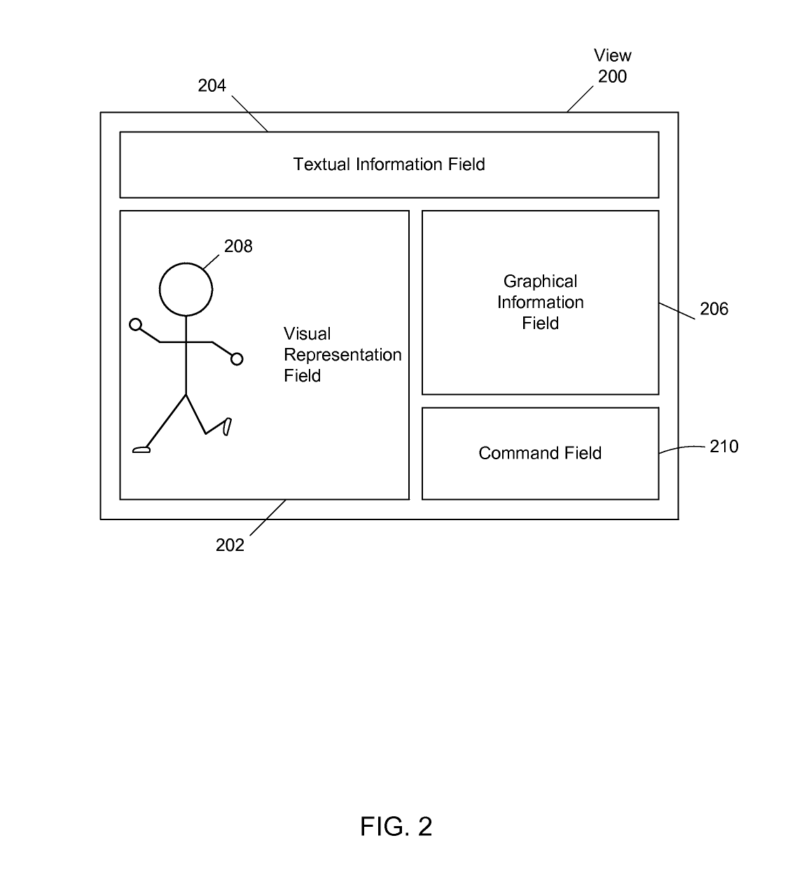

FIG. 2 illustrates a view 200 of information associated with the body positions, physical characteristics, and/or movement of a user, in accordance with one or more implementations. View 200 may be associated with the body positions, physical characteristics, and/or movement of the first user and may be presented to the first user, the second user, and/or other users via client computing platforms 14 (not shown in FIG. 2), for example. In some implementations, view 200 may include a visual representation field 202, a textual information field 204, a graphical information field 206, a command field 210, and/or other fields.

In some implementations, visual representation field 202 may include an avatar 208 of a user and/or other visual information. In some implementations, avatar 208 may represent a target visual representation and/or and actual visual representation, as described above. Textual information field 204 may include, for example, a textual listing of parameters determined by parameterization module 24 and/or other textual information. Graphical information field 206 may include graphs, meters, dials, and/or other graphical information associated with the body position, physical characteristics, and/or movement of the first user. Command field 210 may facilitate entry and/or selection of information for communication between the first user and other users. In some implementations, command field 210 may facilitate entry and/or selection of information that facilitates control of a target visual representation of the first user.

The description of the information presented by the different fields 202, 204, 206, and/or 210 described herein is for illustrative purposes, and is not intended to be limiting, as any of fields 202, 204, 206, and/or 210, and/or other fields may provide more or less information than is described. For example, one or more of fields 202, 204, 206, and/or 210 may be eliminated, and some or all of its information may be provided by other ones of fields 202, 204, 206, 210, and/or other fields not shown in FIG. 2.

Returning to FIG. 1, in some implementations, virtualization module 50 may be configured to provide information related to a three dimensional (3D) rendering of one or more body positions for presentation to a user via a client computing platform 14. In some implementations, a 3D rendering engine may run on a client computing platform 14. The 3D rendering engine may be configured to produce and/or display sequences of renderings representing views of a body position in at least a portion of a virtual three dimensional space. Each rendering may include a snap-shot of a virtual space that the rendering engine produces based on a target, a point of view, the data defining a three dimensional objects' physical properties and motion, parameter information related to a user, and/or other information.

In some implementations, virtualization module 50 may be configured to provide presentation information for presentation to the first user and/or the other users on one or more client computing platforms 14 by implementing an instance of a virtual space. The instance of the virtual space may include a simulated space that is accessible by users via clients (e.g., client computing platforms 14) that present the views of the virtual space to a user. By way of a non-limiting example, the virtual space may simulate a room at a gym for an exercise class. The first user, the second user, and/or other users may participate in the instance of the virtual space (e.g., an exercise class) by controlling one or more user controlled elements in the virtual space. The user controlled elements may include avatars representing the users, for example. Control may be exercised through control inputs and/or commands input by the users through body position sensor 12, client computing platforms 14, and/or other components of system 10.

The presentation information may include data related to views of the virtual space. Virtualization module 50 may be further configured, for example, to send parametric data to a client computing platform 14. The client computing platform 14 may be configured to generate output (e.g., views, sounds) for presentation to the user based on the virtual space data, the parametric data, and/or other data. Virtualization module 50 may be configured to generate presentation information based on information determined by parameterization module 24, user module 44, analysis module 48, modules related to implementing a virtual space (not shown in FIG. 1), other servers, and/or other modules.

Referring again to client communication module 26, in some implementations, client communication module 26 is configured to facilitate receiving the presentation information (e.g., from server 16) for presentation to a given user (e.g., the first user, the second user, and/or other users) via a given client computing platform (e.g., client computing platform 14) associated with the given user. Client presentation module 28 may be configured to facilitate presentation of the received presentation information on client computing platform 14 via user interface 32, and/or other devices. For example, client presentation module 28 may be configured to generate a three dimensional rendering based on the presentation information.

In some implementations, client presentation module 28 may be configured such that users may manipulate views of the presented information. The users may manipulate the presented information via gestures and/or other information entered into user interface 32. For example, via a touchscreen that may be included in user interface 32, users may manipulate a three dimensional rendering that shows a target body position, and/or an actual body position of the first user. The users (including the first user) may use a pinch gesture and/or a reverse pinch gesture to adjust a zoom level of the rendering; one or more swiping motions to tilt and/or rotate the rendering; tap gestures to view, select, and/or deselect one or more portions of the rendering; one or more gestures that allow a user to move a portion of the rendering relative to the rest of the rendering; and/or other control gestures. Manipulating the presented information may allow users to better view and/or understand the presented information. For example, a user may manipulate an arm in the three dimensional rendering of an actual body position of the first user to match a target body position for the arm. The manipulation may demonstrate a motion necessary to achieve the target position.

In some implementations, server communication module 46 and/or client communication module 26 may be configured to facilitate bi-directional communication between client computing platforms 14 associated with the first user and the one or more second users. Server communication module 46 and/or client communication module 26 may be configured such that the first user and/or the one or more second users may communicate information related to body parts, movements and/or other actions by the first user, parameters, and/or other information to each other. Server communication module 46 and/or client communication module 26 may be configured such that communication between the first user and the one or more second users may be via client computing platforms 14, and/or other devices. Communications may be received and entered by the users via their respective client computing platforms 14. Communications may be routed to and from the appropriate users through server 16. For example the communications may be routed through client communication module 26, server communication module 46, and/or virtualization module 50.

By way of a non-limiting example, the users may interact with each other during the exercise class described above through communications exchanged within the virtual space. Such communications may include one or more of textual chat, instant messages, private messages, voice communications, and/or other communications.

In some implementations, server communication module 46 and/or client communication module 26 may be configured such that a second user (e.g., a coach) may communicate to the first user that the first user should perform a specific action. Syntactically, the communication language may allow the coach, in essence, to communicate by forming sentences using one or more of the first user's body parts as nouns, the coach's target body positions and/or movements as verbs, and one or more body position parameters as adjectives and/or adverbs. By way of a non-limiting example, the coach may use system 10 to communicate to the first user that he should raise his arms to form an angle of 90° with his body. In this example, the first user's arms are the nouns, the coach's “raise” command is the verb, and the angle of 90° is an adverb.

External resources 60 may include sources of information, hosts and/or providers of virtual environments outside of system 10, external entities participating with system 10, and/or other resources. In some implementations, some or all of the functionality attributed herein to external resources 60 may be provided by resources included in system 10.

As shown in FIG. 1, user interface 32 may be configured to provide an interface between client computing platform 14 and the user through which the user may provide information to and receive information from system 10. This enables data, cues, results, and/or instructions and any other communicable items, collectively referred to as “information,” to be communicated between the user and system 10. Examples of interface devices suitable for inclusion in user interface 32 include a touch screen, a keypad, buttons, switches, a keyboard, knobs, levers, a display, speakers, a microphone, an indicator light, an audible alarm, a printer, and/or other interface devices. In some implementations, user interface 32 includes a plurality of separate interfaces. In some implementations, user interface 32 includes at least one interface that is provided integrally with client computing platform 14.

It is to be understood that other communication techniques, either hard-wired or wireless, are also contemplated by the present disclosure as user interface 32. For example, the present disclosure contemplates that user interface 32 may be integrated with a removable storage interface provided by client computing platform 14. In this example, information may be loaded into client computing platform 14 from removable storage (e.g., a smart card, a flash drive, a removable disk) that enables the user to customize the implementation of client computing platform 14. Other exemplary input devices and techniques adapted for use with client computing platform 14 as user interface 32 include, but are not limited to, an RS-232 port, RF link, an IR link, modem (telephone, cable or other). In short, any technique for communicating information with client computing platform 14 is contemplated by the present disclosure as user interface 32.

Server 16 may include electronic storage 40, one or more processors 42, and/or other components. Server 16 may include communication lines, or ports to enable the exchange of information with a network and/or other computing platforms (e.g., client computing platforms 14). The illustration of server 16 in FIG. 1 is not intended to be limiting. Server 16 may include a plurality of hardware, software, and/or firmware components operating together to provide the functionality attributed herein to server 16. For example, server 16 may be implemented by a cloud of computing platforms operating together as server 16.

Server 16, body position sensor 12, client computing platforms 14, and/or external resources 60 may be operatively linked via one or more electronic communication links. For example, such electronic communication links may be established, at least in part, via a network such as the Internet and/or other networks. In some implementations, body position sensor 12 and/or external resources 60 may be configured to communicate directly with server 16. It will be appreciated that this is not intended to be limiting, and that the scope of this disclosure includes implementations in which body position sensor 12, server 16, client computing platforms 14, and/or external resources 60 may be operatively linked via some other communication media, or with linkages not shown in FIG. 1. In some implementations, client computing platform 14, body position sensor 12, server 16, and/or other devices may be integrated as a singular device. In some implementations, server 16, client computing platform 14, and/or body position sensor 12 may be integrated as a singular device in the personal wellness device incorporated by reference above.

Electronic storage 34 and/or electronic storage 40 may include electronic storage media that electronically stores information. The electronic storage media of electronic storage 34 may include one or both of system storage that is provided integrally (i.e., substantially non-removable) with client computing platform 14 and/or removable storage that is removably connectable to client computing platform 14 via, for example, a port (e.g., a USB port, a firewire port) or a drive (e.g., a disk drive). The electronic storage media of electronic storage 40 may include one or both of system storage that is provided integrally with server 16 and/or removable storage that is removably connectable to server 16. Electronic storage 34 and/or electronic storage 40 may include one or more of optically readable storage media (e.g., optical disks), magnetically readable storage media (e.g., magnetic tape, magnetic hard drive, floppy drive), electrical charge-based storage media (e.g., EEPROM, RAM), solid-state storage media (e.g., flash drive), and/or other electronically readable storage media. Electronic storage 34 and/or electronic storage 40 may include one or more virtual storage resources (e.g., cloud storage, a virtual private network, and/or other virtual storage resources). Electronic storage 34 and/or electronic storage 40 may store software algorithms, information determined by processor 20 and/or processor 42, information received from server 16, information received from client computing platforms 14, and/or other information that enables client computing platform 14 and/or server 16 to function as described herein. By way of a non-limiting example, the presentation information generated by virtualization module 50 may be stored in electronic storage 40. The stored presentation information may be stored for later presentation to the first user and/or the second user via an associated client computing platform 14.

Processor(s) 20 and/or processor(s) 42 are configured to provide information processing capabilities in system 10. As such, processor 20 and/or processor 42 may each include one or more of a digital processor, an analog processor, a digital circuit designed to process information, an analog circuit designed to process information, a state machine, and/or other mechanisms for electronically processing information. Although processor 20 and processor 42 are each shown in FIG. 1 as a single entity, this is for illustrative purposes only. In some implementations, processor 20 may include a plurality of processing units. Processor 42 may include a plurality of processing units. These processing units may be physically located within the same device, or processors 20 and/or 42 may represent processing functionality of a plurality of devices operating in coordination. Processor 20 may be configured to execute modules 22, 24, 26, 28, and/or other modules. Processor 42 may be configured to execute modules 44, 46, 48, 50, and/or other modules. Processors 20 and/or 42 may be configured to execute modules 22, 24, 26, 28, 44, 46, 48, 50 and/or other modules by software; hardware; firmware; some combination of software, hardware, and/or firmware; and/or other mechanisms for configuring processing capabilities on processor 42.

It should be appreciated that although modules 22, 24, 26, and 28, are illustrated in FIG. 1 as being co-located within a single processing unit, in implementations in which processor 20 includes multiple processing units, one or more of modules 22, 24, 26, and/or 28 may be located remotely from the other modules. Although modules 44, 46, 48, and 50 are illustrated in FIG. 1 as being co-located within a single processing unit, in implementations in which processor 42 includes multiple processing units, one or more of modules 44, 46, 48, and/or 50 may be located remotely from the other modules. The description of the functionality provided by the different modules 22, 24, 26, 28, 44, 46, 48, and/or 50 described herein is for illustrative purposes, and is not intended to be limiting, as any of modules 22, 24, 26, 28, 44, 46, 48, 50 and/or other modules may provide more or less functionality than is described. For example, one or more of modules 22, 24, 26, 28, 44, 46, 48, 50, and/or other modules may be eliminated, and some or all of its functionality may be provided by other ones of modules 22, 24, 26, 28, 44, 46, 48, 50, and/or other modules. As another example, processor 20 and/or processor 42 may be configured to execute one or more additional modules that may perform some or all of the functionality attributed below to one of modules 22, 24, 26, 28, 44, 46, 48, and/or 50.

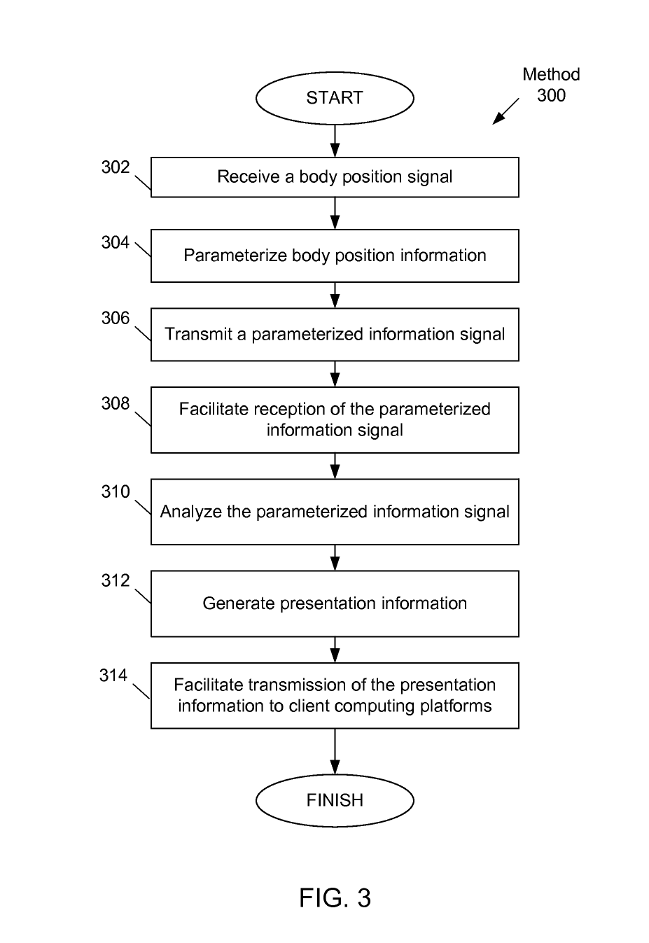

FIG. 3 illustrates a method 300 for determination, communication, and presentation of information related to body positions, in accordance with one or more implementations. The operations of method 300 presented below are intended to be illustrative. In some implementations, method 300 may be accomplished with one or more additional operations not described, and/or without one or more of the operations discussed. Additionally, the order in which the operations of method 300 are illustrated in FIG. 3 and described below is not intended to be limiting.

In some implementations, method 300 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 300 in response to instructions stored electronically on an electronic storage medium. The one or more processing devices may include one or more devices configured through hardware, firmware, and/or software to be specifically designed for execution of one or more of the operations of method 300.

At an operation 302, a body position signal may be received. The body position signal may convey body position information associated with one or more body positions of a first user. The body position signal may be received directly or indirectly from a body position sensor associated with the first user. The information provided by the body position sensor may comprise visual information representing the first user, and/or other information. In some implementations, the body position sensor may be included in a client computing platform associated with the first user. A given body position may describe a spatial position of one or more body parts of the first user. The body position signal may be received responsive to physical activity performed by the first user. In some implementations, operation 302 may be performed by one or more processors configured to execute a computer program module similar to or the same as body recognition module 22 (shown in FIG. 1, and described herein).

At an operation 304, the body position information may be parameterized. The body position information associated with the first user may be parameterized to provide parameterized body position information. The parameterized body position information may comprise less bytes than the body position information conveyed by the body position signal. In some implementations, the parameterized body position information may convey an absolute position, a relative position, a coordinate position, an angle, a geographic location, a velocity, a force, a timing, a duration, a distance, a respiration rate, an effort, a route, a pace, a heart rate, a number of steps, a trajectory, a type of exercise, and/or other parameters. In some implementations, operation 304 may be performed by one or more processors configured to execute a computer program module similar to or the same as parameterization module 24 (shown in FIG. 1, and described herein).

At an operation 306, a parameterized information signal may be transmitted. In some implementations, the parameterized information signal may be transmitted to a server. The parameterized information signal may convey the parameterized body position information. The parameterized body position information may be used to generate presentation information for presentation to one or more other users including a second user. The second user may be remotely located from the first user. The parameterized information signal conveying the parameterized body position information may be transmitted in real-time, near real-time, and/or at a later time. In some implementations, operation 306 may be performed by one or more processors configured to execute a computer program module similar to or the same as client communication module 26 (shown in FIG. 1, and described herein).

At an operation 308, reception of the parameterized information signal may be facilitated. In some implementations, operation 308 may be performed by one or more processors configured to execute a computer program module similar to or the same as server communication module 46 (shown in FIG. 1 and described herein).

At an operation 310, the parameterized information signal may be analyzed. The analysis may determine the physical activity performed by the first user, the effort of the first user during the physical activity, one or more body positions of the first user relative to one or more corresponding target body positions, the route of the first user during the physical activity, and/or other information. In some implementations, operation 310 may be performed by one or more processors configured to execute a computer program module similar to or the same as analysis module 48 (shown in FIG. 1 and described herein).

At an operation 312, presentation information may be generated. The presentation information may be generated for presentation to the first user, the second user, and/or other users and/or systems. The presentation information may be generated based on the received parameterized information signal. The presentation information may include (i) a visual representation of the one or more body positions of the first user, (ii) textual information associated with the one or more body positions of the first user, (iii) graphical information associated with the one or more body positions of the first user, and/or other information. In some implementations, the visual representation of the one or more body positions of the first user may comprise an avatar configured to represent the first user by expressing one or more of the body positions of the first user and/or movement of the first user. In some implementations, operation 312 may be performed by one or more processors configured to execute a computer program module similar to or the same as virtualization module 50 (shown in FIG. 1 and described herein).

At an operation 314, transmission of the presentation information to client computing platforms may be facilitated. The presentation information may be transmitted to the first client computing platform associated with the first user, a second client computing platform associated with the second user, and/or other client computing platforms. The presentation information may be transmitted for presentation to the first user, the second user, and/or other users via the client computing platforms. In some implementations, the presentation information may be transmitted in real-time, near real-time, and/or at a later time. In some implementations, operation 314 may be performed by one or more processors configured to execute a computer program module similar to or the same as server communication module 46 (shown in FIG. 1 and described herein).

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured for determination, communication, and presentation of information related to body positions, the system comprising:
one or more processors configured to execute computer program modules, the computer program modules comprising:
a body recognition module configured to receive a body position signal conveying body position information associated with one or more body positions of a first user, the body position signal being received directly or indirectly from a body position sensor associated with the first user, a given body position describing a spatial position of one or more body parts of the first user, the body position signal being received responsive to physical activity performed by the first user;
a parameterization module configured to parameterize the body position information associated with the first user to provide parameterized body position information, the parameterized body position information comprising less bytes than the body position information conveyed by the body position signal; and
a communication module configured to transmit a parameterized information signal conveying the parameterized body position information, the parameterized body position information being used to generate presentation information for presentation to one or more other users including a second user, the second user being remotely located from the first user, the presentation information including one or more of (i) a visual representation of the one or more body positions of the first user, (ii) textual information associated with the one or more body positions of the first user, or (iii) graphical information associated with the one or more body positions of the first user.

2. The system of claim 1, wherein the parameterized body position information conveys one or more of an absolute position, a relative position, a coordinate position, an angle, a geographic location, a velocity, a force, a timing, a duration, a distance, a respiration rate, an effort, a route, a pace, a heart rate, a number of steps, a trajectory, or a type of exercise.

3. The system of claim 1, wherein the information provided by the body position sensor comprises visual information representing the first user.

4. The system of claim 1, wherein the body position sensor is included in a client computing platform associated with the first user.

5. The system of claim 4, wherein the client computing platform associated with the first user is carried by the first user.

6. The system of claim 1, wherein the communication module is further configured to facilitate receiving the presentation information for presentation to the first user via a client computing platform associated with the first user.

7. The system of claim 6, wherein the client computing platform associated with the first user is configured to generate a three dimensional rendering based on the presentation information for presentation to the first user.

8. The system of claim 1, wherein the communication module is configured to transmit the parameterized information signal conveying the parameterized body position information in real-time, near real-time, and/or at a later time.

9. The system of claim 1, wherein the parameterized body position information conveys information used to identify the physical activity performed by the first user.

10. A system configured for determination, communication, and presentation of information related to body positions, the system comprising:
one or more processors configured to execute computer program modules, the computer program modules comprising:
a communication module configured to facilitate receiving a parameterized information signal conveying parameterized body position information associated with one or more body positions of a first user, the parameterized body position information being based on information conveyed by a body position signal that is received directly or indirectly from a body position sensor associated with the first user, the parameterized body position information comprising less bytes than the body position information conveyed by the body position signal, a given body position describing a spatial position of one or more body parts of the first user, the body position signal being received responsive to physical activity performed by the first user; and
a virtualization module configured to generate presentation information for presentation to one or more other users including a second user, the second user being remotely located from the first user, the presentation information generated based on the received parameterized information signal, the presentation information including one or more of (i) a visual representation of the one or more body positions of the first user, (ii) textual information associated with the one or more body positions of the first user, or (iii) graphical information associated with the one or more body positions of the first user,
wherein the communication module is configured to facilitate transmission of the presentation information to a first client computing platform associated with the first user and/or a second client computing platform associated with the second user for presentation to one or both of the first user or the second user via the first client computing platform and/or the second client computing platform.

11. The system of claim 10, wherein the computer program modules further comprise an analysis module configured to analyze the received parameterized information signal to determine one or more of the physical activity performed by the first user, the effort of the first user during the physical activity, or the route of the first user during the physical activity, and
wherein the virtualization module is configured such that the presentation information includes information related to the analysis of the received parameterized information signal.

12. The system of claim 10, wherein the virtualization module is configured such that the visual representation of the one or more body positions of the first user comprises an avatar configured to represent the first user by expressing one or more of the body positions of the first user and/or movement of the first user.

13. The system of claim 12, wherein the communication module is configured such that facilitating reception of the parameterized information signal and transmission of the presentation information includes facilitating participation in an exercise class conducted in a virtual space via the avatar representing the first user.

14. The system of claim 10, wherein the communication module is configured to facilitate real-time transmission of the presentation information, near real-time transmission of the presentation information, and/or transmission of the presentation information at a later time.

15. The system of claim 10, wherein the communication module is configured to facilitate bi-directional communication between the first client computing platform associated with the first user and the second client computing platform associated with the second user.

16. The system of claim 10, wherein the virtualization module is configured such that the presentation information includes information related to a three dimensional rendering that represents the one or more body positions of the first user for presentation to one or both of the first user via the first client computing platform associated with the first user or the second user via the second client computing platform associated with the second user.

17. A method for determination, communication, and presentation of information related to body positions, the method comprising:
facilitating reception of a parameterized information signal conveying parameterized body position information associated with one or more body positions of a first user, the parameterized body position information being based on information conveyed by a body position signal that is received directly or indirectly from a body position sensor associated with the first user, the parameterized body position information comprising less bytes than the body position information conveyed by the body position signal, a given body position describing a spatial position of one or more body parts of the first user, the body position signal being received responsive to physical activity performed by the first user;
generating presentation information for presentation to one or more other users including a second user, the second user being remotely located from the first user, the presentation information generated based on the received parameterized information signal, the presentation information including one or more of (i) a visual representation of the one or more body positions of the first user, (ii) textual information associated with the one or more body positions of the first user, or (iii) graphical information associated with the one or more body positions of the first user; and
facilitating transmission of the presentation information to a first client computing platform associated with the first user and/or a second client computing platform associated with the second user for presentation to one or both of the first user or the second user via the first client computing platform and/or the second client computing platform.

18. The method of claim 17, further comprising analyzing the received parameterized information signal to determine one or more of the physical activity performed by the first user, the effort of the first user during the physical activity, or the route of the first user during the physical activity, and
wherein the presentation information includes information related to the analysis of the received parameterized information signal.

19. The method of claim 17, wherein the visual representation of the one or more body positions of the first user comprises an avatar configured to represent the first user by expressing one or more of the body positions of the first user and/or movement of the first user.

20. The method of claim 19, wherein facilitating reception of the parameterized information signal and transmission of the presentation information includes facilitating participation in an exercise class conducted in a virtual space via the avatar representing the first user.

21. The method of claim 17, further comprising facilitating real-time transmission of the presentation information, near real-time transmission of the presentation information, and/or transmission of the presentation information at a later time.

22. The method of claim 17, further comprising facilitating bi-directional communication between the first client computing platform associated with the first user and the second client computing platform associated with the second user.

23. The method of claim 17, wherein the presentation information includes information related to a three dimensional rendering that represents the one or more body positions of the first user for presentation to one or both of the first user via the first client computing device associated with the first user or the second user via the second client computing device associated with the second user.
